(19) United States
(12) Patent Application Publication
(10) Pub. No.: US 2024/0246241 A1
(43) Pub. Date: Jul. 25, 2024

(54) INFORMATION PROCESSING APPARATUS, INFORMATION PROCESSING SYSTEM, AND INFORMATION PROCESSING METHOD

(71) Applicant: SONY GROUP CORPORATION, TOKYO (JP)

(72) Inventor: SATOSHI OZAKI, TOKYO (JP)

(21) Appl. No.: 18/561,270

(22) PCT Filed: Mar. 1, 2022
(86) PCT No.: PCT/JP2022/008495

§ 371 Date: Nov. 15, 2023

(30) Foreign Application Priority Data
Jun. 3, 2021 (JP) 2021-093796

(51) Int. Cl. B25J 9/16; G06T 7/73

(52) U.S. Cl. CPC B25J 9/1697; B25J 9/1664; G06T 7/74; G06T 2207/10152; G06T 2207/30004

(57) ABSTRACT

To autonomously control a robot arm at a higher speed. An information processing apparatus according to an embodiment includes a position/posture recognition unit (22) configured to recognize a position and a posture of a surgical tool on the basis of event data input from an event-based vision sensor (EVS) including a plurality of pixels that detects a change in luminance of light from a light emitting unit provided in the surgical tool as an event, and a control instruction unit (23) configured to generate control information for controlling a robot arm that supports a medical device including the EVS on the basis of the recognized position and posture of the surgical tool.

### FIELD

The present disclosure relates to an information processing apparatus, an information processing system, and an information processing method.

### BACKGROUND

In recent years, a surgical system that autonomously controls a surgical instrument (hereinafter, referred to as a surgical tool) such as an endoscope and an electric scalpel with a robot arm, or the like, has been developed.

### CITATION LIST

### Patent Literature

Patent Literature 1: JP 2017-508499 A

### SUMMARY

### Technical Problem

By the way, in autonomous control of a robot arm according to the related art, for example, an image sensor that acquires image data at a predetermined frame rate of about 60 frames per second (fps) has been used. Thus, there is a limit to a recognition speed for autonomously controlling the robot arm, and it has been difficult to autonomously control the robot arm at a higher speed.

Thus, the present disclosure proposes an information processing apparatus, an information processing system, and an information processing method capable of controlling a robot arm at a higher speed.

### Solution to Problem

To solve the problems described above, an information processing apparatus according to an embodiment of the present disclosure includes: a position/posture recognition unit configured to recognize a position and a posture of a surgical tool, on a basis of event data input from an event-based vision sensor (EVS) including a plurality of pixels that detects a change in luminance of light from a light emitting unit provided in the surgical tool as an event; and a control instruction unit configured to generate control information for controlling a robot arm that supports a medical device including the EVS on a basis of the recognized position and posture of the surgical tool.

### BRIEF DESCRIPTION OF DRAWINGS

FIG. 1 is a schematic diagram illustrating a schematic configuration example of an endoscope system according to a first embodiment of the present disclosure.

FIG. 2 is a block diagram illustrating a functional configuration of the endoscope system illustrated in FIG. 1.

FIG. 3 is a schematic diagram illustrating a configuration example of an endoscope according to the first embodiment of the present disclosure.

FIG. 4 is a schematic diagram illustrating a schematic configuration example of a surgical tool according to the first embodiment of the present disclosure.

FIG. 5 is a schematic diagram illustrating a schematic configuration example of a surgical tool according to a modification of the first embodiment of the present disclosure.

FIG. 6 is a schematic diagram illustrating a schematic configuration example of a surgical tool according to another modification of the first embodiment of the present disclosure.

FIG. 7 is a block diagram illustrating a schematic configuration example of an image sensor according to the first embodiment of the present disclosure.

FIG. 8 is a block diagram illustrating a schematic configuration example of an EVS according to the first embodiment of the present disclosure.

FIG. 9 is a waveform diagram illustrating an example of a light emission pattern for each surgical tool according to the first embodiment of the present disclosure.

FIG. 10 is a view for explaining a search pixel range according to the first embodiment of the present disclosure.

FIG. 11 is a view for explaining a pixel region in which an event is detected according to the first embodiment of the present disclosure.

FIG. 12 is a flowchart indicating an operation example of a light emitting apparatus according to the first embodiment of the present disclosure.

FIG. 13 is a flowchart indicating an operation example of an information processing apparatus according to the first embodiment of the present disclosure.

FIG. 14 is a flowchart indicating an operation example of an endoscope control apparatus 40 according to the first embodiment of the present disclosure.

FIG. 15 is a schematic diagram illustrating a schematic configuration example of an endoscope system according to a second embodiment of the present disclosure.

FIG. 16 is a block diagram illustrating a functional configuration of the endoscope system illustrated in FIG. 15.

FIG. 17 is a schematic diagram illustrating a schematic configuration example of an endoscope system according to a third embodiment of the present disclosure.

FIG. 18 is a block diagram illustrating a functional configuration of the endoscope system illustrated in FIG. 17.

FIG. 19 is a waveform diagram illustrating an example of a light emission pattern for each surgical tool according to a modification of the third embodiment of the present disclosure.

FIG. 20 is a hardware configuration diagram illustrating an example of a computer that implements functions of the information processing apparatus according to the present disclosure.

### DESCRIPTION OF EMBODIMENTS

Embodiments of the present disclosure will be described in detail below with reference to the drawings. In each of the following embodiments, the same parts are denoted by the same reference numerals, and redundant description will be omitted.

The present disclosure will be described according to the following item order.
0. Introduction
1. First Embodiment
   1.1 Schematic configuration example of endoscope system
   1.2 Configuration example of endoscope
   1.3 Configuration example of surgical tool
      1.3.1 Modification of surgical tool
   1.4 Configuration example of image sensor
   1.5 Configuration example of EVS
   1.6 Operation example of endoscope system
      1.6.1 Example of light emission pattern for each surgical tool
      1.6.2 Narrowing down of search range
      1. 6. 3 Flowchart
         1.6.3.1 Operation example of light emitting apparatus 50
         1.6.3.2 Operation example of information processing apparatus 20
         1.6.3.3 Operation example of endoscope control apparatus 40
   1.7 Operation and effect
2. Second Embodiment
3. Third Embodiment
   3.1 Modification
4. Hardware configuration

### 0. Introduction

For example, in the field of a surgical system using an autonomous robot arm, in order to autonomously control a position and a posture of a robot arm that supports a medical device such as an endoscope and a surgical microscope or a surgical tool such as an electric scalpel, it is necessary to specify an accurate position, posture, type, and the like, of the surgical tool by acquiring information (for example, image data) regarding a position, a posture, a type, and the like, of the surgical tool used by a surgeon, or the like, and performing processing of recognizing the information. For example, in an endoscope robot that autonomously controls a position and a posture of an endoscope with a robot arm, in order to display a treatment portion to a surgeon, or the like, in an easy-to-understand manner, it is required to accurately recognize a position and a type of a surgical tool used by the surgeon at a high speed and to precisely control the robot arm.

However, an image sensor (hereinafter, also referred to as an RGB sensor) generally used in autonomous control of a robot arm is configured to acquire image data at a predetermined frame rate of, for example, about 60 frames per second (fps), and thus, there is a limit to a recognition speed for autonomously controlling the robot arm, so that it has been difficult to autonomously control the robot arm at a higher speed. Note that, in recent years, a high-speed camera capable of imaging at a high frame rate has been developed, but the high-speed camera itself is large, and thus, it has been difficult to mount the camera in the endoscope supported by the robot arm.

Furthermore, a surface of a surgical tool such as a scalpel and forceps used in surgery is not usually decorated, and thus, it has been difficult to specify an accurate position, posture, type, and the like, of the surgical tool from an image acquired by a normal image sensor. In addition, depending on a surgical method, a surgical tool having a similar shape is used or a plurality of surgical tools of the same type is used, and thus, it has been difficult to specify an accurate position, posture, type, and the like, of each surgical tool from an image acquired by a normal image sensor.

Thus, in the following embodiments, an information processing apparatus, an information processing system, and an information processing method capable of improving a control speed of a robot arm are proposed. In addition, in some or all of the following embodiments, an information processing apparatus, an information processing system, and an information processing method capable of preventing decrease in control accuracy of a robot arm are proposed.

### 1. First Embodiment

First, an information processing apparatus, an information processing system, and an information processing method according to a first embodiment will be described in detail with reference to the drawings. Note that, in the present embodiment, a surgical system (hereinafter, referred to as an endoscope system) that autonomously controls an endoscope with a robot arm will be described as an example of the information processing system. However, the technology according to the present disclosure is not limited to the endoscope system and can be applied to various medical systems such as a microscope system that controls a position and a posture of a surgical microscope with a robot arm, and a surgical system that controls a surgical tool such as an electric scalpel with a robot arm.

### 1.1 Schematic Configuration Example of Endoscope System

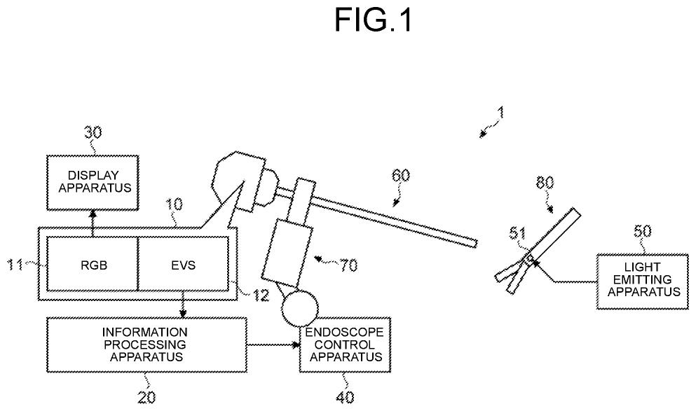

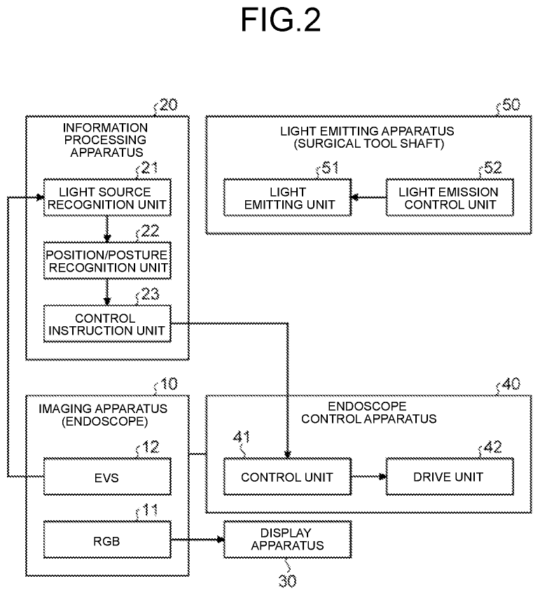

FIG. 1 is a schematic diagram illustrating a schematic configuration example of an endoscope system according to the first embodiment. FIG. 2 is a block diagram illustrating a functional configuration of the endoscope system illustrated in FIG. 1.

As illustrated in FIGS. 1 and 2, an endoscope system 1 includes an imaging apparatus 10, an information processing apparatus 20, a display apparatus 30, an endoscope control apparatus 40, a light emitting apparatus 50, an endoscope 60, a robot arm 70, and a surgical tool 80.

### (Imaging Apparatus 10)

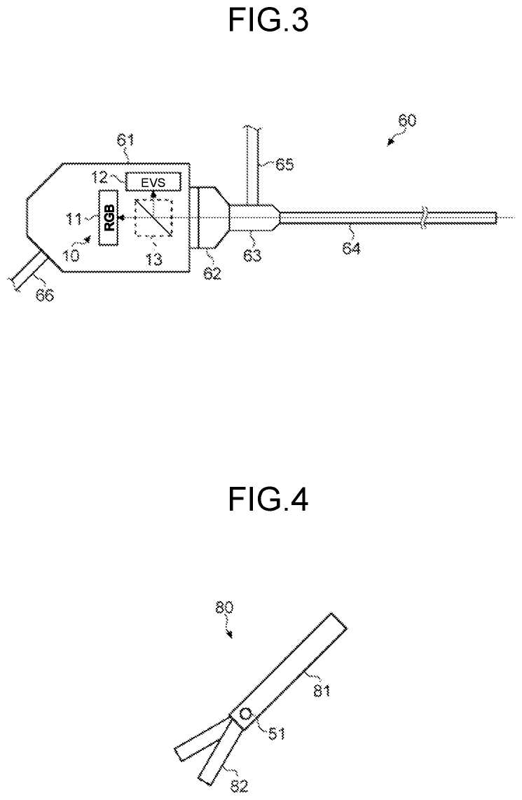

The imaging apparatus 10 includes, for example, an image sensor 11 and an event-based vision sensor (EVS) 12 and is disposed in a camera head 61 (see FIG. 3) of the endoscope 60.

Although described in detail later, the image sensor 11, for example, acquires color image data (hereinafter, referred to as RGB image data) at a predetermined frame rate (for example, 60 fps). The acquired RGB image data is input to the display apparatus 30.

Although details will be described later, the EVS 12, for example, detects an event for each pixel on the basis of a change in luminance of incident light and generates event data indicating content of the detected event. The generated event data is sequentially output to the information processing apparatus 20 as an event stream. On the other hand, the information processing apparatus 20 generates image data (hereinafter, referred to as event image data) based on an event detected for each pixel. Note that a frame rate at which the information processing apparatus 20 generates the event image data may be, for example, equal to or higher than the frame rate of the image sensor 11 (for example, 1000 fps).

### (Information Processing Apparatus 20)

The information processing apparatus 20 includes, for example, a personal computer, or the like, and includes a light source recognition unit 21, a position/posture recognition unit 22, and a control instruction unit 23.

For example, the light source recognition unit 21 specifies a light emission pattern of a light emitting unit 51 included in the event image data by executing recognition processing on the event image data based on the event data input from the EVS 12 of the imaging apparatus 10.

The position/posture recognition unit 22 recognizes a position and a posture of the surgical tool 80 to which the light emitting unit 51 is attached from the event image data for each light emission pattern of the light emitting unit 51 specified by the light source recognition unit 21. The recognition of the position and the posture of the surgical tool 80 is not limited to recognition processing such as pattern matching, and for example, machine learning such as deep neural network (DNN), convolutional neural network (CNN), or recurrent neural network (RNN) may be used. In that case, the event image data may be input to a learned learning model, and the position and the posture of the surgical tool 80 may be output as an estimation result.

The control instruction unit 23 calculates the position and the posture of the endoscope 60 for capturing an image to be displayed on the display apparatus 30 on the basis of the position and the posture of the surgical tool 80 specified by the position/posture recognition unit 22. Then, the control instruction unit 23 generates control information for setting the position and the posture of the endoscope 60 to the calculated position and posture and outputs the generated control information to the endoscope control apparatus 40.

### (Display Apparatus 30)

The display apparatus 30, which includes, for example, a display, or the like, displays an image acquired by the image sensor 11 to a surgical staff such as a surgeon, a scopist and a nurse.

### (Endoscope Control Apparatus 40)

The endoscope control apparatus 40, which includes, for example, an information processing apparatus such as a micro processor unit (MPU), includes a control unit 41 and a drive unit 42.

The control unit 41 generates a drive signal for driving the robot arm 70 on the basis of the control information input from the information processing apparatus 20 and inputs the generated drive signal to the drive unit 42.

The drive unit 42 controls the position and the posture of the endoscope 60 by driving the robot arm 70 according to the drive signal input from the control unit 41.

Note that the endoscope control apparatus 40 may control a magnification (zoom-in/zoom-out) of the image data acquired by the imaging apparatus 10 by controlling an optical system in the endoscope 60 on the basis of the control information input from the information processing apparatus 20.

### (Light Emitting Apparatus 50)

The light emitting apparatus 50 includes a light emitting unit 51 and a light emission control unit 52.

The light emitting unit 51, which includes, for example, a light emitting element such as a light emitting diode (LED), is provided in the surgical tool 80 such as an electric scalpel. For example, in order to suppress influence on RGB image data acquired by the image sensor 11, the light emitting unit 51 preferably outputs light (for example, infrared light or near infrared light) having a wavelength other than a visible light range. However, the present disclosure is not limited thereto, and the light emitting unit 51 may output visible light.

In the present embodiment, the light emission control unit 52 causes the light emitting unit 51 to blink according to a preset light emission cycle.

Note that the light emission control unit 52 may have a configuration separated from the surgical tool 80 and may be connected to the light emitting unit 51 provided in the surgical tool 80 in a wired or wireless manner or may be provided in the surgical tool 80 together with the light emitting unit 51.

### 1.2 Configuration Example of Endoscope

FIG. 3 is a schematic diagram illustrating a configuration example of the endoscope according to the present embodiment. As illustrated in FIG. 3, the endoscope 60 includes, for example, a camera head 61, a merging portion 63, a scope 64, and a joint portion 62.

The scope 64 has a configuration in which an optical fiber passes through a cylindrical tube made of a metal such as stainless steel, and has a configuration in which a lens is provided at a distal end. A rear end of the scope 64 is fixed to the joint portion 62 via the merging portion 63.

An optical fiber cable 65 for guiding irradiation light output from the light emitting unit 51 is inserted into the merging portion 63 from the side. The optical fiber cable 65 passes through the scope 64 from the merging portion 63 and is guided to the distal end of the scope 64. Thus, the irradiation light output from the light emitting unit 51 is emitted from the distal end of the scope 64 via the optical fiber cable 65.

The joint portion 62 is configured to be detachable from the camera head 61, for example. Light incident on the distal end of the scope 64 propagates through an optical fiber (optical fiber different from the optical fiber cable 65) in the scope 64 and is guided to the inside of the camera head 61.

Inside the camera head 61, a spectroscope 13 is provided in addition to the image sensor 11 and the EVS 12 constituting the imaging apparatus 10. As the spectroscope 13, for example, in a case where the light emitting unit 51 outputs light having a wavelength other than the visible light range, an optical element such as a prism or a dichroic mirror that demultiplexes incident light according to the wavelength may be used. On the other hand, in a case where the light emitting unit 51 outputs light having a wavelength other than the visible light range, a half mirror, or the like, may be used as the spectroscope 13.

In the camera head 61, the image sensor 11 and the EVS 12 are arranged such that, for example, planes including the respective light receiving surfaces are substantially perpendicular to each other. In the example illustrated in FIG. 3, the light passing through the spectroscope 13 enters the image sensor 11, and the light reflected by the spectroscope 13 enters the EVS 12. In other words, in the present embodiment, the image sensor 11 and the EVS 12 share the same optical axis. In this event, a coordinate system of the RGB image data acquired by the image sensor 11 and a coordinate system of the event image data acquired by the EVS 12 may be aligned by aligning an angle of view of the image sensor 11 and an angle of view of the EVS 12.

The image data acquired by each of the image sensor 11 and the EVS 12 is output to the outside via a transmission cable 66 provided in the camera head 61. Note that the RGB image data acquired by the image sensor 11 may be directly input to and displayed on the display apparatus 30 or may be input to and displayed on the display apparatus 30 after being temporarily input to the information processing apparatus 20 and subjected to predetermined processing.

### 1.3 Configuration Example of Surgical Tool

FIG. 4 is a schematic diagram illustrating a schematic configuration example of the surgical tool according to the present embodiment. Note that, in this example, a scalpel is exemplified as the surgical tool. As illustrated in FIG. 4, the surgical tool 80 includes, for example, a shaft portion 81 held by a surgeon, or the like, with his/her hand, and a treatment portion 82 provided at a distal end of the shaft portion 81 and performing treatment such as cutting on an affected part, or the like. With respect to such a surgical tool 80, the light emitting unit 51 is provided at a specific position in the shaft portion 81. For example, in a case where the surgical tool 80 is a scalpel, the light emitting unit 51 may be disposed at a position (In FIG. 4, a connection portion between the treatment portion 82 and the shaft portion 81) where a position and a direction of the treatment portion 82 to be opened and closed can be specified.

### 1.3.1 Modification of Surgical Tool

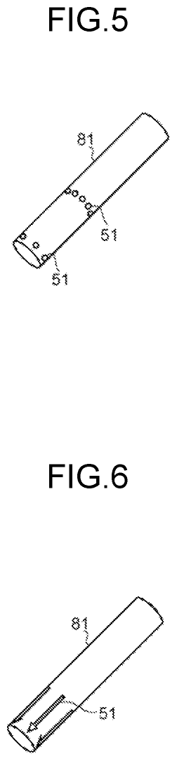

Note that the number of the light emitting units 51 provided in each surgical tool 80 is not limited to one, and may be, for example, two or more as illustrated in FIG. 5. In this event, the arrangement of the light emitting units 51 may be determined so that the type, the position, and the posture of the surgical tool 80 can be specified from the arrangement of the light emitting units 51 detected as an event at the EVS 12. For example, as illustrated in FIG. 5, in a case where the plurality of light emitting units 51 is arranged in a double ring shape with respect to a side surface of the shaft portion 81 in the surgical tool 80, by changing the number of the light emitting units 51 disposed on a distal end side and the number of the light emitting units 51 disposed on a terminal end side, which side is the distal end side where the treatment portion 82 is disposed can be specified from the event image data.

However, the present disclosure is not limited thereto, and for example, as illustrated in FIG. 6, a shape of the light emitting unit 51 may be a shape capable of specifying a direction so that which side is the distal end side on which the treatment portion 82 is disposed can be specified from the event image data.

### 1.4 Configuration Example of Image Sensor

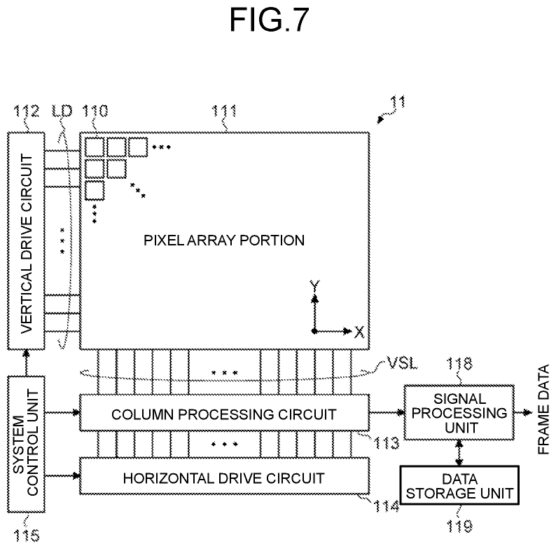

FIG. 7 is a block diagram illustrating a schematic configuration example of the image sensor according to the first embodiment. Note that, in the present example, a complementary metal-oxide-semiconductor (CMOS) image sensor is exemplified, but the present disclosure is not limited thereto, and various image sensors capable of acquiring color or monochrome image data, such as a charge-coupled device (CCD) image sensor, may be used. Furthermore, the CMOS image sensor may be an image sensor created by applying or partially using a CMOS process.

As illustrated in FIG. 7, the image sensor 11 has, for example, a stack structure in which a semiconductor chip on which a pixel array portion 111 is formed and a semiconductor chip on which a peripheral circuit is formed are stacked. The peripheral circuit may include, for example, a vertical drive circuit 112, a column processing circuit 113, a horizontal drive circuit 114, and a system control unit 115.

The image sensor 11 further includes a signal processing unit 118 and a data storage unit 119. The signal processing unit 118 and the data storage unit 119 may be provided on the same semiconductor chip as the peripheral circuit or may be provided on different semiconductor chips.

The pixel array portion 111 has a configuration in which pixels 110 each having a photoelectric conversion element that generates and accumulates a charge according to an amount of received light are arranged in a row direction and a column direction, that is, in a two-dimensional lattice shape in a matrix. Here, the row direction refers to an arrangement direction of pixels in a pixel row (lateral direction in drawings), and the column direction refers to an arrangement direction of pixels in a pixel column (longitudinal direction in drawings).

In the pixel array portion 111, a pixel drive line LD is wired along the row direction for each pixel row, and a vertical signal line VSL is wired along the column direction for each pixel column with respect to the matrix pixel array. The pixel drive line LD transmits a drive signal for driving when a signal is read out from a pixel. In FIG. 7, the pixel drive lines LD are illustrated as wirings one by one, but are not limited to wirings one by one. One end of the pixel drive line LD is connected to an output terminal corresponding to each row of the vertical drive circuit 112.

The vertical drive circuit 112 includes a shift register, an address decoder, and the like, and drives each pixel of the pixel array portion 111 at the same time for all pixels or in units of rows. In other words, the vertical drive circuit 112 constitutes a drive unit that controls operation of each pixel of the pixel array portion 111 together with the system control unit 115 that controls the vertical drive circuit 112. Although a specific configuration of the vertical drive circuit 112 is not illustrated, the vertical drive circuit generally includes two scanning systems of a readout scanning system and a sweep scanning system.

In order to read out a signal from the pixel 110, the readout scanning system sequentially selects and scans the pixel 110 of the pixel array portion 111 in units of rows. The signal to be read out from the pixel 110 is an analog signal. The sweep scanning system performs sweep scanning on a readout row on which readout scanning is to be performed by the readout scanning system prior to the readout scanning by an exposure time.

By the sweep scanning by the sweep scanning system, unnecessary charges are swept out from photoelectric conversion elements of the pixels 110 in the readout row, whereby the photoelectric conversion elements are reset. Then, by sweeping out (resetting) unnecessary charges in the sweep scanning system, so-called electronic shutter operation is performed. Here, the electronic shutter operation refers to operation of discarding charges of the photoelectric conversion elements and newly starting exposure (starting accumulation of charges).

The signal to be read out by readout operation by the readout scanning system corresponds to an amount of light received after the immediately preceding readout operation or electronic shutter operation. Then, a period from a readout timing by the immediately preceding readout operation or a sweep timing by the electronic shutter operation until a readout timing by the current readout operation is a charge accumulation period (also referred to as an exposure period) in the pixel 110.

A signal to be output from each pixel 110 of the pixel row selectively scanned by the vertical drive circuit 112 is input to the column processing circuit 113 through each of the vertical signal lines VSL for each pixel column. The column processing circuit 113 performs predetermined signal processing on the signal output from each pixel of the selected row through the vertical signal line VSL for each pixel column of the pixel array portion 111 and temporarily holds a pixel signal after the signal processing.

Specifically, the column processing circuit 113 performs at least noise removal processing, for example, correlated double sampling (CDS) processing or double data sampling (DDS) processing, as the signal processing. For example, fixed pattern noise unique to the pixel such as reset noise and threshold variation of an amplification transistor in the pixel is removed by the CDS processing. The column processing circuit 113 also has, for example, an analog-digital (AD) conversion function, converts an analog pixel signal read out from the photoelectric conversion element into a digital signal and outputs the digital signal.

The horizontal drive circuit 114 includes a shift register, an address decoder, and the like, and sequentially selects a readout circuit (hereinafter, referred to as a pixel circuit) corresponding to a pixel column of the column processing circuit 113. By selective scanning by the horizontal drive circuit 114, pixel signals subjected to signal processing for each pixel circuit in the column processing circuit 113 are sequentially output.

The system control unit 115 includes a timing generator that generates various timing signals, and the like, and performs drive control of the vertical drive circuit 112, the column processing circuit 113, the horizontal drive circuit 114, and the like, on the basis of various timings generated by the timing generator.

The signal processing unit 118 has at least an arithmetic processing function and performs various kinds of signal processing such as arithmetic processing on the pixel signals output from the column processing circuit 113. The data storage unit 119 temporarily stores data necessary for signal processing in the signal processing unit 118.

Note that the RGB image data output from the signal processing unit 118 may be directly input to and displayed on the display apparatus 30 as described above or may be input to and displayed on the display apparatus 30 after being temporarily input to the information processing apparatus 20 and subjected to predetermined processing.

### 1.5 Configuration Example of EVS

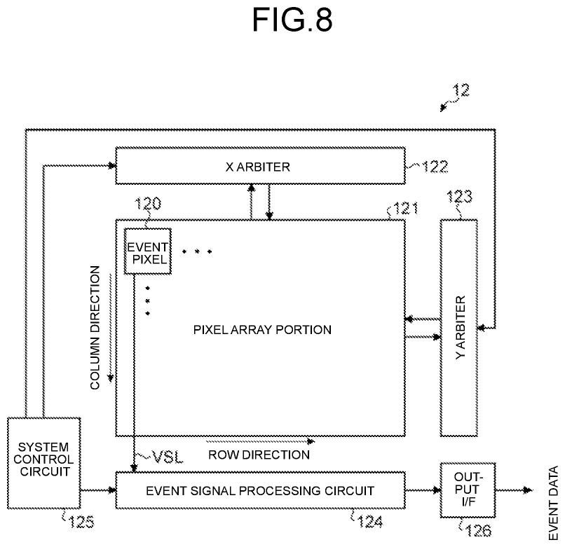

Next, a schematic configuration example of the EVS 12 will be described. FIG. 8 is a block diagram illustrating a schematic configuration example of the EVS according to the present embodiment. As illustrated in FIG. 8, the EVS 12 includes a pixel array portion 121, an X arbiter 122 and a Y arbiter 123, an event signal processing circuit 124, a system control circuit 125, and an output interface (I/F) 126.

The pixel array portion 121 has a configuration in which a plurality of event pixels 120 that detects an event on the basis of a luminance change of incident light is arranged in a two-dimensional lattice pattern. Note that, in the following description, a row direction (also referred to as a row direction) refers to an arrangement direction of pixels in a pixel row (lateral direction in drawings), and a column direction (also referred to as a column direction) refers to an arrangement direction of pixels in a pixel column (longitudinal direction in drawings).

Each event pixel 120 includes a photoelectric conversion element that generates a charge according to luminance of the incident light. In a case where a change in luminance of the incident light is detected on the basis of a photocurrent flowing out from the photoelectric conversion element, the event pixel outputs a request for requesting readout from the event pixel to the X arbiter 122 and the Y arbiter 123 and outputs an event signal indicating that an event has been detected according to arbitration by the X arbiter 122 and the Y arbiter 123.

Each event pixel 120 detects presence or absence of an event on the basis of whether or not a change exceeding a predetermined threshold has occurred in the photocurrent according to the luminance of the incident light. For example, each event pixel 120 detects that a change in luminance exceeds a predetermined threshold value (positive event) or falls below the predetermined threshold value (negative event) as an event.

When an event is detected, the event pixel 120 outputs a request for requesting permission to output an event signal indicating occurrence of the event to each of the X arbiter 122 and the Y arbiter 123. Then, the event pixel 120 outputs an event signal to the event signal processing circuit 124 in a case where a response indicating permission to output an event signal is received from each of the X arbiter 122 and the Y arbiter 123.

The X arbiter 122 and the Y arbiter 123 arbitrate the request for requesting the output of the event signal supplied from each of the plurality of event pixels 120, and transmit a response based on the arbitration result (permission/non-permission of the output of the event signal) and a reset signal for resetting event detection to the event pixel 120 that has output the request.

The event signal processing circuit 124 generates and outputs event data by executing predetermined signal processing on the event signal input from the event pixel 120.

As described above, a change in the photocurrent generated in the event pixel 120 can also be regarded as a change of a light amount (change in luminance) of the light incident on the photoelectric conversion unit of the event pixel 120. Thus, it can also be said that the event is a change of the light amount (change in luminance) of the event pixel 120 exceeding the predetermined threshold. The event data indicating occurrence of the event includes at least position information such as a coordinate indicating the position of the event pixel 120 where the change of the light amount as the event has occurred. The event data can include the polarity of the change of the light amount in addition to the position information.

For a series of event data to be output at a timing when an event has occurred from the event pixel 120, as long as the interval between the pieces of event data is maintained at the time when the event has occurred, it can be said that the event data implicitly includes time information indicating relative time when the event has occurred.

However, if the interval between the pieces of event data is not maintained as it is at the time of occurrence of the event due to the event data being stored in a memory, or the like, the time information implicitly included in the event data is lost. Thus, before the interval between the pieces of event data is not maintained as it is at the time of occurrence of the event, the event signal processing circuit 124 may include time information indicating relative time at which the event has occurred, such as a time stamp in the event data.

### (Other Configurations)

The system control circuit 125 includes a timing generator that generates various kinds of timing signals, and the like, and performs drive control of the X arbiter 122, the Y arbiter 123, the event signal processing circuit 124, and the like, on the basis of various kinds of timings generated by the timing generator.

The output I/F 126 sequentially outputs the event data output in units of rows from the event signal processing circuit 124 to the information processing apparatus 20 as an event stream. On the other hand, the information processing apparatus 20 (for example, the light source recognition unit 21 or an event data processing unit (not illustrated)) accumulates the event data input as the event stream for a predetermined frame period to generate event image data of a predetermined frame rate.

### 1.6 Operation Example of Endoscope System

Next, an operation example of the endoscope system 1 according to the present embodiment will be described. In the present embodiment, control of the robot arm 70 is executed on the basis of the event image data acquired by the EVS 12. Thus, in the present embodiment, in order to enable specification of not only the position and the posture of each surgical tool 80 but also each of individual surgical tools 80 and a type of each of the surgical tools 80 from the event image data, the light emitting unit 51 is caused to blink in a light emission pattern unique to each of the surgical tools 80 or unique to the type of each of the surgical tools 80.

### 1.6.1 Example of Light Emission Pattern for Each Surgical Tool

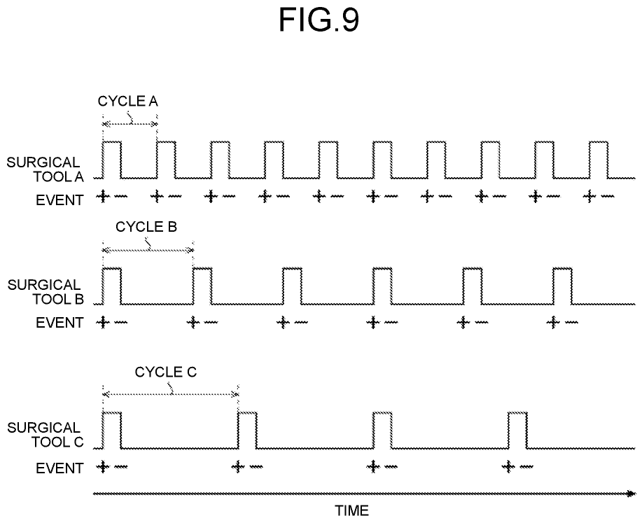

FIG. 9 is a waveform diagram illustrating an example of a light emission pattern for each surgical tool according to the present embodiment. Note that, in the present description, a case where three surgical tools 80 of the surgical tools A to C are used will be exemplified. The surgical tools A to C may be the same type of the surgical tools 80 or may be different types of the surgical tools 80.

As illustrated in FIG. 9, the light emitting unit 51 attached to the surgical tool A emits light (hereinafter, also referred to as “light emission of surgical tool A”) in, for example, a light emission pattern in which turning on and off are repeated in a cycle A (this is referred to as a light emission pattern A). For example, the light emitting unit 51 attached to the surgical tool B emits light (hereinafter, also referred to as “light emission of surgical tool B”) in a light emission pattern in which turning on and off are repeated in a cycle B different from the cycle A (this is referred to as a “light emission pattern B”). For example, the light emitting unit 51 attached to the surgical tool C emits light (hereinafter, also referred to as “light emission of the surgical tool C”) in a light emission pattern in which turning on and off are repeated in a cycle C different from the cycles A and B (this is referred to as a “light emission pattern C”). In order to reduce overlapping of light emission periods of the surgical tools A to C, the cycles A to C are preferably cycles that are not in a relationship of a multiple or a divisor.

The event pixel 120 in which an image of the light emitting unit 51 of each of the surgical tools A to C in the EVS 12 is formed detects a positive event and generates event data (hereinafter, also referred to as positive event data) at a timing when each of the surgical tools A to C is turned on, that is, at a timing when the drive signal supplied to the light emitting unit 51 rises and detects a negative event and generates event data (hereinafter, also referred to as negative event data) at a timing when each of the surgical tools A to C is turned off, that is, at a timing when the drive signal supplied to the light emitting unit 51 falls.

Thus, the light source recognition unit 21 in the information processing apparatus 20 can specify which of the surgical tools A to C a region where each event has occurred corresponds to by specifying a cycle of occurrence of the positive event or the negative event from the event image data input at a predetermined frame rate. Note that the position and the posture of each of the surgical tools A to C can be specified from, for example, the position and the posture of the endoscope 60 and the position, shape, distribution, or the like, of the region where each event has occurred in the event image data.

Furthermore, specification of the individual surgical tools 80 or the types thereof by the light emission pattern can also be performed, for example, by calculating a difference (luminance difference) between frames of the RGB image data acquired by the image sensor 11. However, by using the event image data instead of the RGB image data, not only the individual surgical tools 80 or the types thereof can be specified at a higher frame rate, but also it is not necessary to calculate a difference between the frames, so that it is possible to execute specification of the individual surgical tools 80 or the types thereof at a higher speed. This means that, even if the frame rate of the event image data is equal to the frame rate of the RGB image data, it is possible to specify the individual surgical tools 80 or the type thereof at a higher speed.

### 1.6.2 Narrowing Down of Search Range

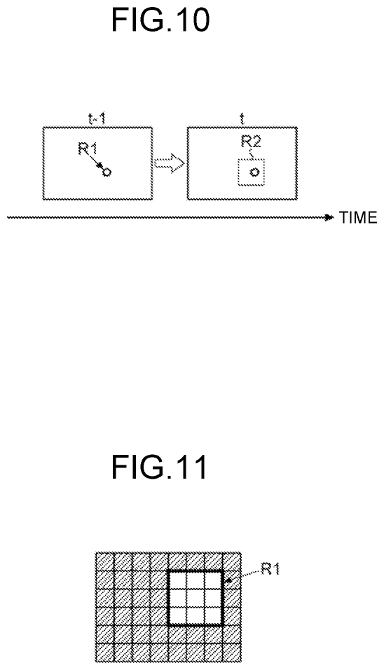

Furthermore, in a case where the surgical tool 80 is recognized using the EVS 12 as in the present embodiment, the EVS 12 can capture a change in luminance at an extremely high speed (for example, 1000 fps, or the like), and thus, even if a position of the surgical tool 80 moves, a moving distance between frames is expected to be small. Thus, in the present embodiment, as illustrated in FIG. 10, recognition processing in the next and subsequent frames is executed by narrowing down to events generated in a region (hereinafter, a search pixel range R2) in the vicinity of a pixel region R1 where the event has been detected, thereby reducing load and speed of the recognition processing and preventing erroneous recognition.

Here, for example, the search pixel range R2 may be set by calculating movement of an angle of view of the imaging apparatus 10 using optical flow of the entire event image data and movement information of the robot arm 70 using the pixel region R1 in which the event has been detected as a reference. In addition, in a case where the angle of view greatly moves, setting of the search pixel range R2 may be canceled, and the entire event image data may be set as the search target.

Note that, as illustrated in FIG. 11, in a case where the light emitting unit 51 is imaged by the EVS 12, usually, the pixel region R1 in which the event has been detected has a certain extent. Thus, for example, a barycentric coordinate of the pixel region R1, or the like, may be used as a reference position when the search pixel range R2 is set.

Furthermore, the search pixel range R2 may be set and adjusted in consideration of a shape, a size, and the like, of the pixel region R1 in addition to the barycentric coordinate obtained in the current frame. For example, if the light emitting unit 51 is close to a distal end of the scope 64 of the endoscope 60, the image of the light emitting unit 51 in the event image data becomes large, and many pixels detect an event. Conversely, if the light emitting unit 51 is far from the distal end, few pixels detect an event. Thus, the size of the search pixel range R2 may be adjusted according to the distance between the distal end of the scope 64 and the light emitting unit 51.

For measurement of the distance between the distal end of the scope 64 and the light emitting unit 51, for example, if the endoscope 60 performs monaural imaging, a triangulation method by stereo vision using two or more pieces of event image data continuous in time series may be used. Furthermore, in a case where the endoscope 60 performs stereo imaging, a triangulation method by stereo vision using two or more pieces of event image data acquired from respective viewpoints may be used. However, the present disclosure is not limited thereto, and for example, the distance between the distal end of the scope 64 and the light emitting unit 51 may be measured using a distance measuring sensor such as a time of flight (ToF) sensor.

In addition to the distance between the distal end of the scope 64 and the light emitting unit 51, the position of the light emitting unit 51 in the three-dimensional space may be specified. Similarly to the measurement of the distance, a triangulation method, a distance measuring sensor, or the like, can be used to specify the position of the light emitting unit 51 in the three-dimensional space. By specifying the position of the light emitting unit 51 in the three-dimensional space, the surgeon, and the like, can more accurately specify from which angle the surgical tool 80 should be imaged.

### 1.6.3 Flowchart

Next, operation of the light emitting apparatus 50, the information processing apparatus 20, and the endoscope control apparatus 40 will be described in detail with reference to a flowchart.

### 1.6.3.1 Operation Example of Light Emitting Apparatus 50

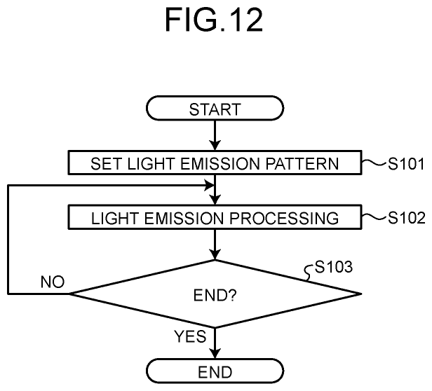

FIG. 12 is a flowchart indicating an operation example of the light emitting apparatus according to the present embodiment. As indicated in FIG. 12, in the present operation, first, the light emission control unit 52 of the light emitting apparatus 50 sets a light emission pattern for each surgical tool 80 or a type thereof (step S101). The light emission pattern for each surgical tool 80 or a type thereof may be set such that, for example, the light emission control unit 52 recognizes the surgical tool 80, and the light emission pattern allocated in advance to the recognized surgical tool 80 is automatically set, or the light emission pattern may be manually set for each surgical tool 80 to be used for surgery by a surgical staff, a mechanical staff, or the like.

If the light emission pattern is set for each surgical tool 80 in this manner, next, the light emission control unit 52 inputs a drive signal to each surgical tool 80 to cause the light emitting unit 51 of each surgical tool 80 to emit light in each light emission pattern (step S102).

Thereafter, the light emission control unit 52 determines whether or not to end the present operation on the basis of end of the surgery, stop of use of the surgical tool 80, and the like, (step S103) and ends the present operation in a case where it is determined to end the operation (step S103: Yes). On the other hand, in a case where it is determined not to end the present operation (step S103: No), the processing of the light emission control unit 52 returns to step S102, and the light emission control unit 52 continues the subsequent operation.

### 1.6.3.2 Operation Example of Information Processing Apparatus 20

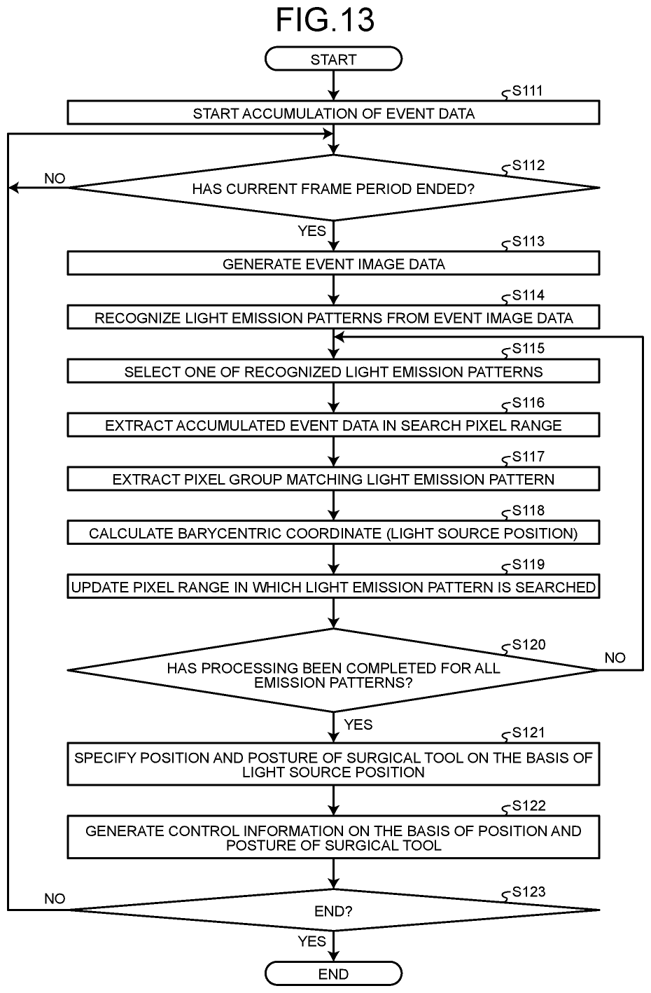

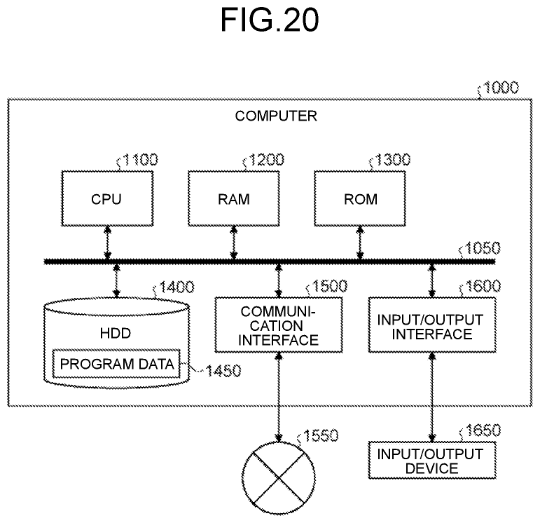

FIG. 13 is a flowchart indicating an operation example of the information processing apparatus according to the present embodiment. Note that, in the present description, attention is paid to operation of a control unit (for example, corresponding to a CPU 1100 in FIG. 20 which will be described later) that controls each unit of the information processing apparatus 20.

As indicated in FIG. 13, in the present operation, first, the control unit of the information processing apparatus 20 starts accumulation of event data irregularly input from the EVS 12 in a memory (for example, a RAM 1200, an HDD 1400, or the like, in FIG. 20 to be described later) not illustrated after activation of the endoscope system 1 (step S111). Note that the accumulated event data may be only positive event data, only negative event data, or both positive event data and negative event data. In addition, each event data may include a time stamp (time), position information (coordinates), and the polarity of a change of the light amount.

Next, the control unit determines whether or not the frame period of the current frame has ended (step S112), and in a case where the frame period of the current frame has ended (step S112: Yes), the control unit generates event image data from the event data accumulated during a current frame period (step S113).

Next, the control unit inputs the generated event image data to the light source recognition unit 21 to recognize the light emission pattern of each pixel in which the event has occurred in the event image data (step S114). For example, in the example illustrated in FIG. 9, the light emission patterns A to C are recognized. Note that recognition of the light emission pattern in this step may use not only the event image data of the current frame but also event image data of one or more previous frames stored in a memory (not illustrated), or the like.

Next, the control unit selects one unselected light emission pattern from the light emission patterns recognized in step S114 (step S115). For example, in the example illustrated in FIG. 9, one unselected light emission pattern is selected from the light emission patterns A to C recognized in step S114.

Next, the control unit extracts event data accumulated for each pixel included in the search pixel range R2 set in the initial setting or the processing for the previous frame (step S116).

Next, on the basis of the extracted event data, the control unit extracts a pixel group matching the light emission pattern selected in step S115 from the pixels included in the search pixel range R2 (step S117) and calculates a barycentric coordinate (light source position) of the extracted pixel group (step S118).

Next, the control unit updates the search pixel range R2 on the basis of the barycentric coordinate calculated in step S118, the shape and the size of the pixel group extracted in step S117, the optical flow of the entire event image data, the movement information of the robot arm 70, and the like (step S119).

Next, the control unit determines whether or not the processing of steps S115 to S119 has been executed for all the light emission patterns recognized in step S114 (step S120), and in a case where an unselected light emission pattern remains (step S120: No), the processing returns to step S115, and the control unit executes the subsequent processing for the unselected light emission pattern.

On the other hand, in a case where the processing for all the light emission patterns has been completed (step S120: Yes), the control unit inputs the barycentric coordinate (and if necessary, the shape and the size of the pixel group extracted in step S117) calculated in step S118 to the position/posture recognition unit 22 to specify the position and the posture of the surgical tool 80 (step S121).

Next, on the basis of the position and the posture of the surgical tool specified in step S121, the control unit generates control information for controlling the endoscope 60 to a position and a posture at which the treatment portion 82 of the surgical tool 80 can fall within the angle of view so that the surgeon, and the like, can easily see (step S122). The generated control information is input to the control unit 41 of the endoscope control apparatus 40. Note that the processing in steps S115 to S122 may be executed by the position/posture recognition unit 22.

Thereafter, the control unit determines whether or not to end the present operation on the basis of end of the surgery, stop of use of the surgical tool 80, and the like, (step S123) and ends the present operation in a case where it is determined to end the operation (step S123: Yes). On the other hand, in a case where it is determined not to end the present operation (step S123: No), the processing of the control unit returns to step S112, and the control unit continues the subsequent operation.

### 1.6.3.3 Operation Example of Endoscope Control Apparatus 40

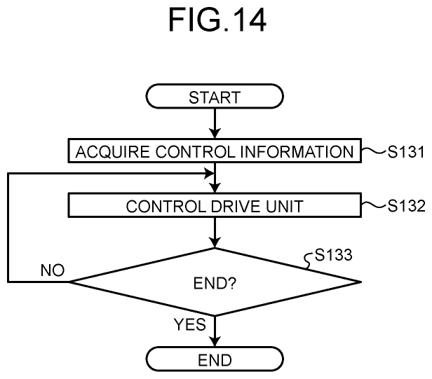

FIG. 14 is a flowchart indicating an operation example of the endoscope control apparatus 40 according to the present embodiment. As indicated in FIG. 14, when the control information is acquired from the information processing apparatus 20 after activation (step S131), the control unit 41 of the endoscope control apparatus 40 generates a drive signal of the robot arm 70 for controlling the endoscope 60 to a desired position and posture on the basis of the acquired control information (step S132). The generated drive signal is supplied to the drive unit 42. As a result, the robot arm 70 is driven, and the position and the posture of the endoscope 60 are controlled to a desired position and posture.

Thereafter, the control unit 41 determines whether or not to end the present operation on the basis of end of the surgery, stop of use of the surgical tool 80, and the like, (step S133) and ends the present operation in a case where it is determined to end the operation (step S133: Yes). On the other hand, in a case where it is determined not to end the present operation (step S133: No), the processing of the control unit returns to step S131, and the control unit continues the subsequent operation.

1.7 Operation and effect

As described above, in the present embodiment, the position and the posture of the surgical tool 80 are recognized using the event image data obtained by the EVS 12 instead of the image data obtained by the image sensor 11, so that it is possible to perform recognition processing at a frame rate higher than a frame rate of a general image sensor. This makes it possible to improve the control speed of the robot arm.

Furthermore, in the present embodiment, the light emitting unit 51 attached to each surgical tool 80 blinks in a unique light emission pattern, and the blinking is detected by the EVS 12, so that it is possible to specify a type, a position, and a posture of each surgical tool 80 at a high speed with a small processing amount. This makes it possible to prevent decrease in control accuracy of the robot arm.

### 2. Second Embodiment

Next, an information processing apparatus, an information processing system, and an information processing method according to a second embodiment of the present disclosure will be described in detail with reference to the drawings. Note that, in the present embodiment, as in the first embodiment, an endoscope system is taken as an example of the information processing system.

In the first embodiment described above, a case where the type of the surgical tool 80 and the position and the posture thereof are recognized using the event image data acquired by the EVS 12 has been exemplified, but the present disclosure is not limited thereto. For example, the type of the surgical tool 80 and the position and the posture thereof may be recognized using RGB image data acquired by the image sensor 11 in addition to the event image data acquired by the EVS 12.

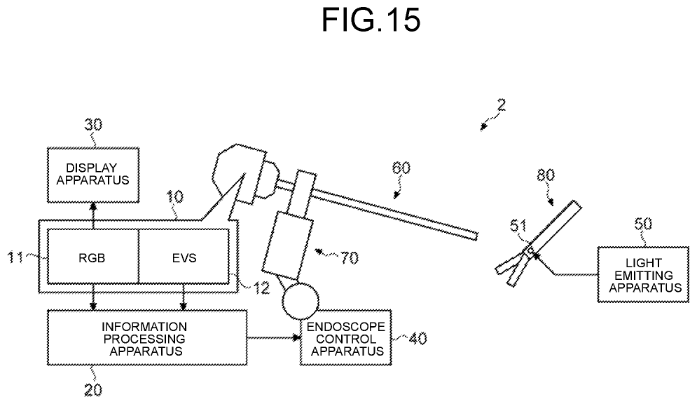

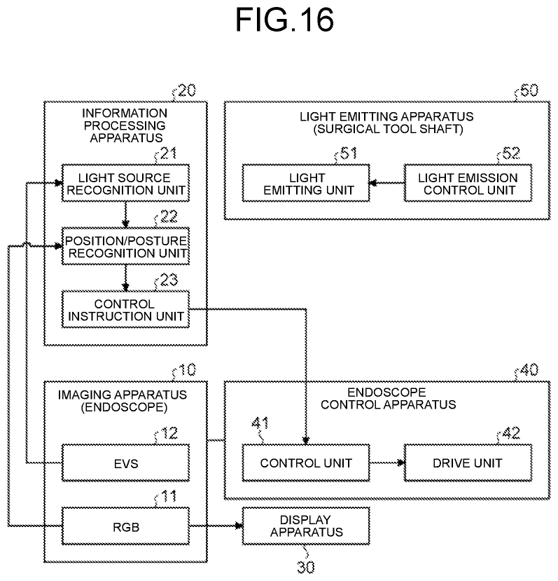

FIG. 15 is a schematic diagram illustrating a schematic configuration example of the endoscope system according to a second embodiment. FIG. 16 is a block diagram illustrating a functional configuration of the endoscope system illustrated in FIG. 15.

As illustrated in FIGS. 15 and 16, an endoscope system 2 according to the present embodiment has a configuration in which RGB image data acquired by the image sensor 11 is input to the position/posture recognition unit 22 of the information processing apparatus 20 in a configuration similar to that of the endoscope system 1 described with reference to FIGS. 1 and 2 in the first embodiment.

The position/posture recognition unit 22 recognizes the position and the posture of the surgical tool 80 to which the light emitting unit 51 is attached from the event image data and the RGB image data for each light emission pattern of the light emitting unit 51 specified by the light source recognition unit 21. The recognition of the position and the posture of the surgical tool 80 is not limited to recognition processing such as pattern matching, and for example, machine learning such as DNN, CNN, or RNN may be used.

In a case where the position and the posture of the surgical tool 80 are recognized by recognition processing such as pattern matching, the position and the posture of the surgical tool 80 recognized from the event image data and the position and the posture of the surgical tool 80 recognized from the RGB image data may be integrated, and the final position and posture of the surgical tool 80 recognized from the image data may be recognized.

On the other hand, in a case where machine learning is used, the event image data and the RGB image data may be input to a learned learning model, and the position and the posture of the surgical tool 80 may be output as an estimation result.

However, in a case where a frame rate of the EVS 12 is higher than a frame rate of the image sensor 11, the event image data and the RGB image data cannot be necessarily used in combination for all the frames. In this case, the event image data and the RGB image data may be used in combination only in a frame in which the event image data and the RGB image data can be used in combination.

Furthermore, in a case where movement of the angle of view of the endoscope 60 is large, blur may occur in RGB image data having a low frame rate. Thus, in the present embodiment, in a case where movement of the angle of view of the endoscope 60 is large, the final position and posture of the surgical tool 80 may be recognized by increasing importance of the position and the posture of the surgical tool 80 obtained from the event image data or the event image data.

In this manner, by recognizing the position and the posture of the surgical tool 80 using the RGB image data in addition to the event image data, the recognition accuracy can be improved. This makes it possible to improve the control accuracy of the robot arm.

Other configurations, operation, and effects may be similar to those of the first embodiment described above, and thus, detailed description thereof will be omitted here.

### 3. Third Embodiment

Next, an information processing apparatus, an information processing system, and an information processing method according to a third embodiment of the present disclosure will be described in detail with reference to the drawings. Note that, in the present embodiment, as in the first and second embodiments, an endoscope system is taken as an example of the information processing system.

In the first and second embodiments described above, light emission of the light emitting unit 51 by the light emitting apparatus 50 and frame data generation of event image data by the information processing apparatus 20 are executed independently. On the other hand, in the third embodiment, a case where light emission of the light emitting unit 51 by the light emitting apparatus 50 is controlled by the information processing apparatus 20 will be exemplified.

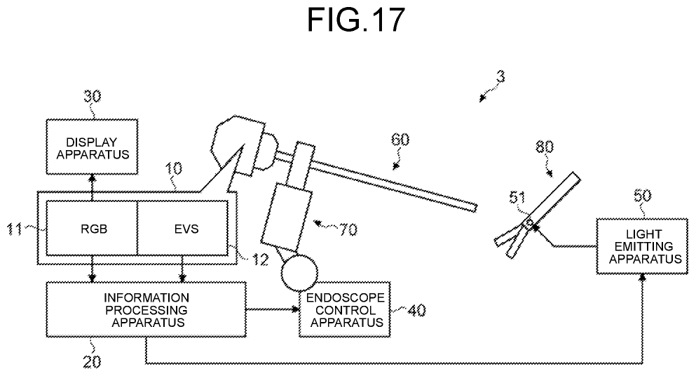

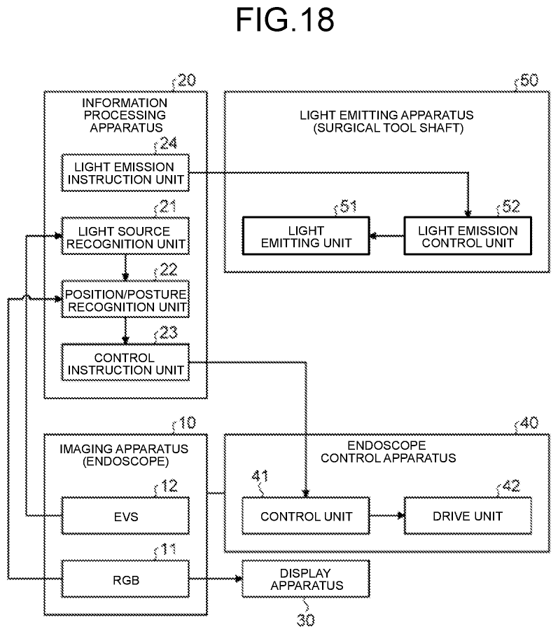

FIG. 17 is a schematic diagram illustrating a schematic configuration example of the endoscope system according to the third embodiment. FIG. 18 is a block diagram illustrating a functional configuration of the endoscope system illustrated in FIG. 17.

As illustrated in FIGS. 17 and 18, an endoscope system 3 according to the present embodiment has a configuration in which the information processing apparatus 20 further includes a light emission instruction unit 24 in a configuration similar to the configuration of the endoscope system 2 described with reference to FIGS. 15 and 16 in the second embodiment. Note that while a configuration based on the endoscope system 2 according to the second embodiment is employed in the present example, the present disclosure is not limited thereto, and for example, a configuration based on the endoscope system 1 according to the first embodiment may be employed.

The light emission instruction unit 24 outputs light emission control information for causing the light emitting unit 51 to emit light in accordance with a generation timing of frame data of the event image data by the information processing apparatus 20 to the light emission control unit 52 of the light emitting apparatus 50. On the other hand, the light emission control unit 52 generates a drive signal for causing the light emitting unit 51 to emit light on the basis of the input light emission control information and inputs the generated drive signal to the light emitting unit 51. As a result, the light emitting unit 51 is controlled to emit light in accordance with the generation timing of the frame data of the event image data by the information processing apparatus 20.

As described above, by making it possible to control the light emission timing of the light emitting unit 51 on the information processing apparatus 20 side, it is possible to cause the light emitting unit 51 of each surgical tool 80 to emit light when necessary on the information processing apparatus 20 side, so that it is possible to more accurately recognize a type, a position, and a posture of the surgical tool 80. For example, in a case where it is desired to display a video focusing on a specific surgical tool 80 such as an electric scalpel on the display apparatus 30, it is possible to more quickly and accurately recognize a position and a posture of the specific surgical tool 80 by selectively causing the specific surgical tool 80 to emit light, so that it is possible to enhance reliability of control of the endoscope 60 by the robot arm 70.

Other configurations, operation, and effects may be similar to those of the first or second embodiment described above, and thus, detailed description thereof will be omitted here.

### 3.1 Modification

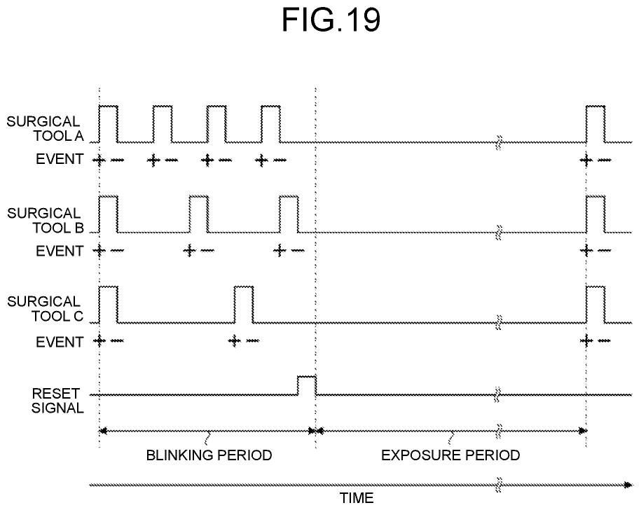

Furthermore, in a case where a light emission timing of the light emitting unit 51 can be controlled on the information processing apparatus 20 side as in the third embodiment, for example, as illustrated in FIG. 19, a blinking period of the light emitting unit 51 may be controlled so that the blinking period of the light emitting unit 51 does not overlap with an exposure period of the image sensor 11. In that case, a reset signal for resetting the charges accumulated in each pixel 110 by photoelectric conversion may be supplied to each pixel 110 of the image sensor 11 at a timing when the blinking period ends, that is, immediately before the exposure period is started.

As described above, by dividing the blinking period of the light emitting unit 51 and the exposure period of the image sensor 11, for example, even in a case where a visible light source is used for the light emitting unit 51, influence of blinking of the light emitting unit 51 on the RGB image data can be minimized.

### 4. Hardware Configuration

The information processing apparatus 20, the endoscope control apparatus 40, and the light emitting apparatus 50 according to the embodiment, the modifications thereof, and the application examples described above can be implemented by a computer 1000 having a configuration as illustrated in FIG. 20, for example. FIG. 20 is a hardware configuration diagram illustrating an example of the computer 1000 that implements the respective functions of the information processing apparatus 20, the endoscope control apparatus 40, and the light emitting apparatus 50. The computer 1000 includes a CPU 1100, a RAM 1200, a read only memory (ROM) 1300, a hard disk drive (HDD) 1400, a communication interface 1500, and an input/output interface 1600. The respective units of the computer 1000 are connected by a bus 1050.

The CPU 1100 operates on the basis of a program stored in the ROM 1300 or the HDD 1400 and controls the respective units. For example, the CPU 1100 develops programs stored in the ROM 1300 or the HDD 1400 in the RAM 1200 and executes processing corresponding to various programs.

The ROM 1300 stores a boot program such as a basic input output system (BIOS) to be executed by the CPU 1100 when the computer 1000 is activated, a program depending on hardware of the computer 1000, and the like.

The HDD 1400 is a computer-readable recording medium that non-transiently records a program to be executed by the CPU 1100, data to be used by the program, and the like. Specifically, the HDD 1400 is a recording medium that records a program for implementing respective kinds of operation according to the present disclosure which is an example of program data 1450.

The communication interface 1500 is an interface for the computer 1000 to connect to an external network 1550 (for example, the Internet). For example, the CPU 1100 receives data from another device or transmits data generated by the CPU 1100 to another device via the communication interface 1500.

The input/output interface 1600 has a configuration including the I/F unit 18 described above and is an interface for connecting an input/output device 1650 and the computer 1000. For example, the CPU 1100 receives data from an input device such as a keyboard and a mouse via the input/output interface 1600. In addition, the CPU 1100 transmits data to an output device such as a display, a speaker, or a printer via the input/output interface 1600. Furthermore, the input/output interface 1600 may function as a media interface that reads a program, or the like, recorded in a predetermined recording medium (medium). The medium is, for example, an optical recording medium such as a digital versatile disc (DVD) or a phase change rewritable disk (PD), a magneto-optical recording medium such as a magneto-optical disk (MO), a tape medium, a magnetic recording medium, a semiconductor memory, or the like.

For example, in a case where the computer 1000 functions as each of the information processing apparatus 20, the endoscope control apparatus 40, and the light emitting apparatus 50 according to the above-described embodiments, the CPU 1100 of the computer 1000 executes a program loaded on the RAM 1200 to implement the respective functions of the information processing apparatus 20, the endoscope control apparatus 40, and the light emitting apparatus 50. In addition, the HDD 1400 stores a program, and the like, according to the present disclosure. Note that the CPU 1100 reads the program data 1450 from the HDD 1400 and executes the program data 1450, but as another example, these programs may be acquired from another apparatus via the external network 1550.

Although the embodiments of the present disclosure have been described above, the technical scope of the present disclosure is not limited to the above-described embodiments as it is, and various modifications can be made without departing from the gist of the present disclosure. In addition, components of different embodiments and modifications may be appropriately combined.

Furthermore, the effects in the respective embodiments described in the present specification are merely examples and are not limited, and other effects may be provided.

Note that the present technology can also have the following configurations.
(1)

An information processing apparatus including:
a position/posture recognition unit configured to recognize a position and a posture of a surgical tool, on a basis of event data input from an event-based vision sensor (EVS) including a plurality of pixels that detects a change in luminance of light from a light emitting unit provided in the surgical tool as an event; and
a control instruction unit configured to generate control information for controlling a robot arm that supports a medical device including the EVS on a basis of the recognized position and posture of the surgical tool.
(2)

The information processing apparatus according to (1),
wherein the position/posture recognition unit recognizes the position and the posture of the surgical tool on a basis of frame data generated from event data input within a predetermined frame period.
(3)

The information processing apparatus according to (2),
wherein the position/posture recognition unit recognizes the position and the posture of the surgical tool on a basis of the event data within a search range set on a basis of frame data of a previous frame.
(4)

The information processing apparatus according to (3), wherein the position/posture recognition unit sets the search range of an event for frame data of a next frame, using a region where the event has occurred in frame data of a current frame as a reference.
(5)

The information processing apparatus according to (4),
wherein the position/posture recognition unit sets the search range further on a basis of at least one of optical flow of the entire frame data of the current frame and movement information of the robot arm.
(6)

The information processing apparatus according to any one of (3) to (5),
wherein, in a case where an angle of view of the EVS greatly moves, the position/posture recognition unit cancels setting of the search range and recognizes the position and the posture of the surgical tool on a basis of the entire frame data.
(7)

The information processing apparatus according to any one of (1) to (6),
wherein the position/posture recognition unit recognizes the position and the posture of the surgical tool by machine learning using the event data as an input.
(8)

The information processing apparatus according to any one of (1) to (7),
wherein the position/posture recognition unit recognizes the position and the posture of the surgical tool further on a basis of image data input from an image sensor that shares an optical axis with the EVS.
(9)

The information processing apparatus according to (8), further including:
a light emission instruction unit configured to generate a light emission control signal for controlling a light emission period of the light emitting unit,
wherein light from the light emitting unit is light in a visible light range, and
the light emission instruction unit generates the light emission control signal for causing the light emitting unit to emit light during a period that does not overlap with an exposure period of the image sensor.
(10)

The information processing apparatus according to any one of (1) to (9), further including:
a light source recognition unit configured to individually specify the surgical tool or a type of the surgical tool on a basis of the event detected in each of the pixels,
wherein the position/posture recognition unit recognizes the position and the posture for each of the surgical tools.
(11)

The information processing apparatus according to (10), further including:
a light emission instruction unit configured to generate a light emission control signal for controlling a light emission pattern of the light emitting unit,
wherein the light source recognition unit specifies a light emission pattern of the light emitting unit on a basis of the event detected in each of the pixels and individually specifies the surgical tool or a type of the surgical tool on a basis of the specified light emission pattern.
(12)

The information processing apparatus according to (11),
wherein the light emission instruction unit generates the light emission control signal such that the light emitting units provided in different surgical tools or different types of surgical tools emit light in different light emission patterns.
(13)

An information processing system including:
a light emitting unit provided in a surgical tool;
a medical device including an EVS including a plurality of pixels that detects a change in luminance of light from the light emitting unit as an event;
an information processing apparatus configured to generates control information for controlling a robot arm that supports the medical device on a basis of event data input from the EVS; and
a control apparatus configured to control the robot arm on a basis of the control information.
(14)

The information processing system according to (13),
wherein the information processing apparatus includes:
a position/posture recognition unit configured to recognize a position and a posture of the surgical tool on a basis of the event data input from the EVS; and
a control instruction unit configured to generate control information for controlling the robot arm that supports the medical device including the EVS on a basis of the recognized position and posture of the surgical tool.
(15)

The information processing system according to (13) or (14),
wherein the medical device is an endoscope or a surgical microscope.
(16)

An information processing method including:
recognizing a position and a posture of a surgical tool on a basis of event data input from an event-based vision sensor (EVS) including a plurality of pixels that detects a change in luminance of light from a light emitting unit provided in the surgical tool as an event; and
generating control information for controlling a robot arm that supports a medical device including the EVS on a basis of the recognized position and posture of the surgical tool.

### REFERENCE SIGNS LIST

1, 2, 3 ENDOSCOPE SYSTEM
10 IMAGING APPARATUS
11 IMAGE SENSOR
12 EVS
13 SPECTROSCOPE
20 INFORMATION PROCESSING APPARATUS
21 LIGHT SOURCE RECOGNITION UNIT
22 POSITION/POSTURE RECOGNITION UNIT
23 CONTROL INSTRUCTION UNIT
24 LIGHT EMISSION INSTRUCTION UNIT
30 DISPLAY APPARATUS
40 ENDOSCOPE CONTROL APPARATUS
41 CONTROL UNIT
42 DRIVE UNIT
50 LIGHT EMITTING APPARATUS
51 LIGHT EMITTING UNIT
52 LIGHT EMISSION CONTROL UNIT
60 ENDOSCOPE
61 CAMERA HEAD
62 JOINT PORTION
63 MERGING PORTION
64 SCOPE
65 OPTICAL FIBER CABLE
66 TRANSMISSION CABLE
70 ROBOT ARM
80 SURGICAL TOOL
81 SHAFT PORTION
82 TREATMENT PORTION

1. An information processing apparatus including:
a position/posture recognition unit configured to recognize a position and a posture of a surgical tool, on a basis of event data input from an event-based vision sensor (EVS) including a plurality of pixels that detects a change in luminance of light from a light emitting unit provided in the surgical tool as an event; and
a control instruction unit configured to generate control information for controlling a robot arm that supports a medical device including the EVS on a basis of the recognized position and posture of the surgical tool.

2. The information processing apparatus according to claim 1,
wherein the position/posture recognition unit recognizes the position and the posture of the surgical tool on a basis of frame data generated from event data input within a predetermined frame period.

3. The information processing apparatus according to claim 2,
wherein the position/posture recognition unit recognizes the position and the posture of the surgical tool on a basis of the event data within a search range set on a basis of frame data of a previous frame.

4. The information processing apparatus according to claim 3,
wherein the position/posture recognition unit sets the search range of an event for frame data of a next frame, using a region where the event has occurred in frame data of a current frame as a reference.

5. The information processing apparatus according to claim 4,
wherein the position/posture recognition unit sets the search range further on a basis of at least one of optical flow of the entire frame data of the current frame and movement information of the robot arm.

6. The information processing apparatus according to claim 3,
wherein, in a case where an angle of view of the EVS greatly moves, the position/posture recognition unit cancels setting of the search range and recognizes the position and the posture of the surgical tool on a basis of the entire frame data.

7. The information processing apparatus according to claim 1,
wherein the position/posture recognition unit recognizes the position and the posture of the surgical tool by machine learning using the event data as an input.

8. The information processing apparatus according to claim 1,
wherein the position/posture recognition unit recognizes the position and the posture of the surgical tool further on a basis of image data input from an image sensor that shares an optical axis with the EVS.

9. The information processing apparatus according to claim 8, further including:
a light emission instruction unit configured to generate a light emission control signal for controlling a light emission period of the light emitting unit,
wherein light from the light emitting unit is light in a visible light range, and
the light emission instruction unit generates the light emission control signal for causing the light emitting unit to emit light during a period that does not overlap with an exposure period of the image sensor.

10. The information processing apparatus according to claim 1, further including:
a light source recognition unit configured to individually specify the surgical tool or a type of the surgical tool on a basis of the event detected in each of the pixels,
wherein the position/posture recognition unit recognizes the position and the posture for each of the surgical tools.

11. The information processing apparatus according to claim 10, further including:
a light emission instruction unit configured to generate a light emission control signal for controlling a light emission pattern of the light emitting unit,
wherein the light source recognition unit specifies a light emission pattern of the light emitting unit on a basis of the event detected in each of the pixels and individually specifies the surgical tool or a type of the surgical tool on a basis of the specified light emission pattern.

12. The information processing apparatus according to claim 11,
wherein the light emission instruction unit generates the light emission control signal such that the light emitting units provided in different surgical tools or different types of surgical tools emit light in different light emission patterns.

13. An information processing system including:
a light emitting unit provided in a surgical tool;
a medical device including an EVS including a plurality of pixels that detects a change in luminance of light from the light emitting unit as an event;
an information processing apparatus configured to generates control information for controlling a robot arm that supports the medical device on a basis of event data input from the EVS; and
a control apparatus configured to control the robot arm on a basis of the control information.

14. The information processing system according to claim 13,
wherein the information processing apparatus includes:
a position/posture recognition unit configured to recognize a position and a posture of the surgical tool on a basis of the event data input from the EVS; and
a control instruction unit configured to generate control information for controlling the robot arm that supports the medical device including the EVS on a basis of the recognized position and posture of the surgical tool.

15. The information processing system according to claim 13,
wherein the medical device is an endoscope or a surgical microscope.

16. An information processing method including:
recognizing a position and a posture of a surgical tool on a basis of event data input from an event-based vision sensor (EVS) including a plurality of pixels that detects a change in luminance of light from a light emitting unit provided in the surgical tool as an event; and
generating control information for controlling a robot arm that supports a medical device including the EVS on a basis of the recognized position and posture of the surgical tool.
